(19) United States
(12) Patent Application Publication
(10) Pub. No.: US 2010/0239678 A1
(43) Pub. Date: Sep. 23, 2010

(54) IONICALLY FUNCTIONALIZED NANODIAMONDS

(76) Inventor: Ali RAZAVI, Dallas, PA (US)

Correspondence Address: ZALE Patent Law, 121 Old Orchard Road, Clarks Summit, PA, 18503-2053, US

(21) Appl. No.: 12/795,587

(22) Filed: Jun. 7, 2010

Related U.S. Application Data
(63) Continuation-in-part of application No. 12/626,614, filed on Nov. 25, 2009.
Application No. 12/626,614 is a continuation-in-part of application No. 12/399,844, filed on Mar. 6, 2009.
Application No. 12/399,844 is a continuation-in-part of application No. 12/301,356, filed on Nov. 18, 2008.
(60) Provisional application No. 61/118,281, filed on Nov. 26, 2008.
(60) Provisional application No. 61/034,173, filed on Mar. 6, 2008.
(60) Provisional application No. 60/831,438, filed on Jul. 18, 2006.

(51) Int. Cl. A61K 9/14; C07D 215/00; C07D 499/44; C07C 205/00; C07C 255/00; A61K 31/47; A61K 31/43; A61K 31/047; A61K 31/54; A61K 31/275; A61K 31/7052; A61K 31/7048

(52) U.S. Cl. 424/489; 546/152; 540/314; 568/712; 558/303; 514/311; 514/192; 514/727; 514/223.8; 514/526; 514/29; 977/773; 977/906

(57) ABSTRACT

The present invention is directed to attaching drugs 11 and other functional groups to surfaces of nano-sized diamond particles (NDs) 20 to enhance their efficacy. The method involves hydrating a plurality of nanodiamond (ND) particles 20 having a plurality of carbon chain surface molecules 23 on its surface. Cations 40 are embedded within the lattice structure 21 of the surface molecules 23 of the ND particles 20. The embedded cations 40 attract anions that cause crystalline growth. The anion form of drug molecules 11 are then grown on the crystal to cause the NDs 20 to be coated such that the active sites of said drug molecules 11 point away from the ND particle 20 exposing them for enhanced activity and enhanced drug efficacy. The efficacy of antimicrobial drugs 11, as well as other drugs 11 are enhanced by their attachment to the NDs 20.

### CROSS REFERENCE TO RELATED APPLICATIONS

This patent application is a Continuation-In-Part patent application of earlier-filed Continuation-In-Part patent application Ser. No. 12/626,614 filed Nov. 25, 2009 entitled “Nanodiamond Enhanced Drugs”, which was a Continuation-In-Part of Ser. No. 12/399,844 filed Mar. 6, 2009 entitled “Nanodiamond Enhanced Efficacy” which claimed priority from provisional patent application Ser. No. 61/034,173 filed Mar. 6, 2008 having the same title.

This Continuation-In-Part patent application also claims priority from provisional patent application Ser. No. 12/626,614 filed Nov. 25, 2009 entitled “Nanodiamond Enhanced Drugs” which claimed priority from 61/118,281 filed Nov. 26, 2008 having the same title.

This is also a Continuation-In-Part patent application of U.S. patent application Ser. No. 12/301,356 entitled “Multifunctional Articles And Method For Making The Same” filed Nov. 18, 2008 which was field from international patent application PCT/US07/16194 filed Jul. 17, 2007 with the same title which claims the benefit of U.S. Provisional Patent Application 60/831,438 entitled “Biofunctional Articles For Personal Care Applications and Method of Making the Same” filed Jul. 18, 2006.

### BACKGROUND OF THE INVENTION

1. Field of the Invention

The present invention relates to a substance and method of enhancing the efficacy of drugs and more specifically a substance and method of increasing the efficacy of antimicrobials (AMs).

2. Discussion of Related Art

There has always been a need to increase the efficacy of various drugs and preparations. One such class of drugs to be used as an example in this application are the antimicrobials (AMs). AMs are typically used to limit or stop the spread of unwanted microbes. AMs are effective when used in the proper level but lose their ability as the concentration drops below a critical concentration. This may be due to the fact that enough active sites on antimicrobial molecules must make contact with active sites on critical molecules inside of a microbe to cause the microbe to have an effect.

AMs are typically used in an aqueous solution inside of a person or animal. Molecules flowing in a solution are randomly dispersed and oriented. Also, since AMs flow in solution to attach to active sites on the microbe, it is important to have a large amount of the AMs in solution, increasing the local concentration and the potential of attaching to an active site on a microbe molecule.

Currently, there is a need for AMs that are more soluble in a fluid, and attach more readily to active sites on molecules of microbes to inactivate the microbes.

### SUMMARY OF THE INVENTION

The present invention may be embodied as a method of enhancing efficacy of a drug entity 11 having an active site 13, comprising the steps of:
a) acquiring a plurality of nanodiamond (ND) particles 20 having a surface with a plurality of carbon chain surface molecules 23 in a crystalline lattice structure 21, the ND particles 20 having a diameter of less than 10 nanometers;
b) attaching water molecules 30 to the surface of the ND particles 20 to prepare said surface for further reactions;
c) embedding cations 40 within the lattice structure 21 of the surface molecules, creating crystalline growth;
d) providing said drug entity 11 in an anionic form to the surface of the ND particles 20 creating ionic bonds with the cations 40 embedded within the lattice structure 21, thereby coating the NDs particles 20 increasing efficacy and solubility of the drug functional groups 11.

The drug entity 11 used can be an anion of one of the group consisting of:
fluoroquinilone, Amoxycillin, 2-bromo-2-nitropropane-1,3-diol, 3,5-dimethyltetrahydro-1,3,5-2H-thiazine-2-thione, N-(trichloromethyl)-thiop-hthalimide, butyl-p-hydroxy-benzoate, diiodomethyl-p-tolysulfone, and tetrachloroisophthalonitrile, azithromycin, penicillin and clarithromycin.
The functionalized ND particles 54 can be incorporated into threads of one of the group consisting of: woven fabrics used in medical industry, clothing having antimicrobial properties, and clothing resistant to microbial growth and unpleasant odors.

The present invention may be embodied as an enhancing efficacy drug entity 11 having an active site 13, created from the process comprising the steps of:
a) acquiring a plurality of nanodiamond (ND) particles 20 having a surface with a plurality of carbon chain surface molecules 23 in a crystalline lattice structure 21, the ND particles 20 having a diameter of less than 10 nanometers;
b) attaching water molecules 30 to the surface of the ND particles 20 to prepare said surface for further reactions;
c) embedding cations 40 within the lattice structure 21 of the surface molecules, creating crystalline growth;
d) providing said drug entity 11 in an anionic form to the surface of the ND particles 20 creating ionic bonds with the cations 40 embedded within the lattice structure 21, thereby coating the NDs particles 20 increasing efficacy and solubility of the drug functional groups 11.

The present invention may be embodied as a method of enhancing the efficacy of a drug entity 11 comprising the steps of:
a) acquiring nanodiamond (ND) particles having carbon chain surface molecules created by a detonation process with the majority of the particles having a diameter of less than 10 nm;
b) processing the surface of the ND particles 20 by hydrating them to create a layer of water molecules on the ND particles 20;
c) embedding cations within the carbon chain surface molecules thereby ‘salting out’ water molecules on the surface of the ND particles 20;
d) providing the drug entity 11 in anionic form to the surface of the ND particles 20 to cause an ionic bond with the cations 40 embedded within the carbon chain surface molecules 23 to result in functionalized ND particles 54 exhibiting enhanced efficacy when compared to prior art drugs.

This method is very effective in enhancing the efficacy of antimicrobial agents; however other drugs may be employed.

### OBJECTS OF THE INVENTION

It is an object of the present invention to provide an antimicrobial which is more potent than previous antimicrobials.

It is another object of the present invention to provide an antimicrobial which is more soluble than prior art antimicrobials.

It is another object of the present invention to provide a method of amplifying the effect of an antimicrobial in-situ.

It is another object of the present invention to provide a method of holding antimicrobial molecules in an orientation to maximize interaction with microbes.

It is another object of the present invention to provide a method for locally increasing the effective concentration of an antimicrobial while keeping the overall concentration constant.

### BRIEF DESCRIPTION OF THE DRAWINGS

The advantages of the instant disclosure will become more apparent when read with the specification and the drawings, wherein:

FIG. 1 is a schematic illustration of how molecules react under normal prior art conditions.

FIG. 2 is a schematic microscopic view of a portion of a nanodiamond showing the structure of chemical entities attached to the surface of the nanodiamond.

FIG. 3 is an illustration of the hydration step of the nano-diamonds surface according to one embodiment of the present invention.

FIG. 4 is an illustration of the crystalline structure of nanodiamond.

FIG. 5 is an illustration of the ‘salting out’ step of the nanodiamond surface according to one embodiment of the present invention.

FIG. 6 is an illustration of a reaction for attaching drug molecules to cations embedded within the surface of the nanodiamond according to the present invention.

FIG. 7 is an illustration of test plates used for testing the effectiveness of the present invention.

FIG. 8 is a graph of Erythema Scores vs. time.

FIG. 9 is a graph of Mean Edema Scores vs. time.

FIG. 10 is a graph of wound size vs. time.

### DETAILED DESCRIPTION OF THE INVENTION

### Theory

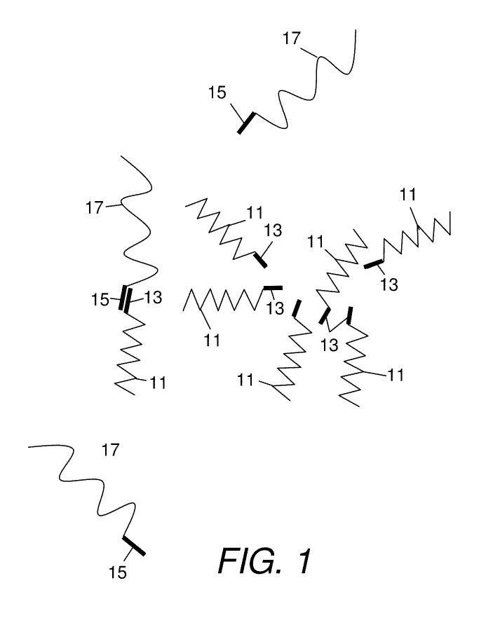

FIG. 1 is a schematic illustration of how molecules react under normal prior art conditions.

As stated in the “Background of the Invention”, chemical functional groups, antimicrobials AM 11 here for this description, typically in solution, randomly orient themselves and by random chance align in the proper orientation to have an active chemical site 13 of the AMs make contact with the proper active chemical site 15 of a molecule in a microbe 17.

If these active sites 13 are hidden inside a clump of molecules 11 (shown in the center of the figure) or otherwise inaccessible, the chances that the active sites 13 make contact another active site 15 of the microbe is reduced. It is better if the active sites are exposed.

When enough of these reactions occur, the reaction may cause the microbe to cease functioning. In one case it may cause them to stop their pathogenic function. In another case, the AMs may kill the microbe. This is the basis for fighting pathogens with antimicrobial chemicals.

These may be by numerous different mechanisms. One such mechanism is to impede a chain reaction which creates cell walls. This causes offspring to have weak cell walls making them more vulnerable to attack by the body's natural defenses such as macrophages.

Other mechanisms attack the microbe's ability to reproduce, or attack the energy producing mitochondria. Some AMs may use a combination of these mechanisms.

Since each of these are based upon the random motion of molecules in solution, the chances that an active site of a molecule having the proper orientation makes contact with an active site of the proper molecule of the microbe is a matter of chance. The greater the number of molecules and active sites in solution, the greater the chances of the desired chemical bindings between active sites. Therefore, by exposing and holding the active sites of the AMs outward in an exposed, fixed orientation and gradually varying the orientations across a surface, there will be an orderly array of exposed active sites.

This may be due to the fact that enough active sites on antimicrobial molecules must make contact with active sites on critical molecules inside of a microbe to cause the microbe to become deactivated.

Molecules flowing in a solution are randomly dispersed and oriented. Also, since AMs flow in solution to attach to active sites on the microbe, it is important to have a large amount of the AMs in solution, increasing the local concentration and the potential of attaching to an active site on a microbe molecule.

Also, the orderly arrangement of active sites must be able to move to meet up with the molecules of the microbe to interact with the active sites of these molecules. Therefore, this orderly arrangement must be mobile.

Foreign objects in the body are identified by the body's immune system and either destroyed or ejected from the body. The immune cells of the body may seek out and kill, or engulf and carry foreign objects out of the body. This would greatly reduce the efficacy of any drug introduced into the body which is recognized as a foreign substance.

The body ignores particles which are 10 Nanometers (nm.) or smaller. This may be due to the fact that there are many naturally occurring objects in the body fluids which are 10 nm. or smaller.

Nanodiamonds (“ND”) are diamonds which are 6 nm or smaller. These are typically produced according to the process explained in U.S. Pat. Nos. 5,916,955 and 5,861,349 assigned to NanoBlox, Inc. issued Jun. 29, 1999 and Jan. 19, 1999, respectively. In this process, carbon is converted in an explosive process to create NDs in which the vast majority of the NDs produced are approximately 6 nm.

These can be cleaned to take any graphite off of the surface to result in pure NDs of about 5 nm in diameter.

NDs have been shown to stabilize suspensions and solutions and greatly increase solubility of substances in solutions.

NDs have also been known to be functionalized to attach fluorine groups to its surface. This was intended to alter the surface composition of the NDs, but not for the purposes similar to that of the present invention.

Since particles of 10 nm or less are allowed to freely pass in and out of microbes, it is believed that these may be perfect transport vehicles for many different AMs. Therefore AMs would be attached to the NDs to create an AM-ND complex.

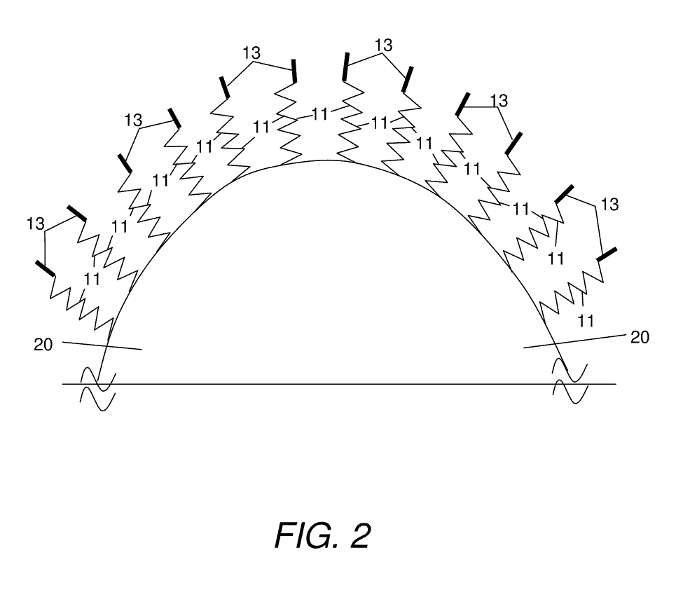

FIG. 2 is a schematic microscopic view of a portion of a nanodiamond showing the structure of chemical entities attached to the surface of the nanodiamond.

The ND 20 exhibits a spherical shape. Here one is covered with a plurality of chemical entities, which are antimicrobial chemical entities, the AMs 11. The AMs 11 are fixed in an orientation which extends them outwardly.

This causes the active sites 13 of each of the AMs 11 to be exposed and point outwardly. Since the surface of ND 20 is curved, as one moves along the surface in any direction, the orientation of the AMs 11 and their active sites 13 changes slightly, allowing a continuum of orientations for the active sites 13. Therefore, there is a greater chance of randomly oriented molecules to come in contact with an active site 13 of Am 11 having the proper orientation for reaction.

Therefore, if one were to supply an orderly arrangement of such AM molecules covering the surface of the NDs with the active sites facing outwardly, it is believed that the efficacy of the AMs would be greatly increased.

It was found, by extensive trial and error, that the efficacy of substances can be amplified by attachment to functionalized NDs.

Therefore, if one were to supply an orderly arrangement of such drug molecules covering the surface of the NDs with the active sites facing outwardly, it is believed that the efficacy of the drugs would be greatly increased.

It was found, that the efficacy of substances can be amplified by attachment to functionalized NDs.

Electrostatic Functionalization of Nanodiamond with Cations and Ionic Crystal Growth.

This wet chemistry procedure has been developed to selectively attach functional groups to the surface of nanodiamond (ND) 20. The functionalized NDs have potential applications in medicine.

The process is based upon three steps:
1. Hydration Step—The exposed carbon chains on the surface of the NDs are prepared for functionalization by hydration.
2. Intermediate Step—cations are incorporated into the interstitial sites of the surface of the NDs. This creates ionic crystalline growth to ‘salt out’ the hydration water on the surface of the NDs.
3. Functionalization Step—Functional groups are attached to the cations of the ionic crystalline growth in the interstitial spaces of the NDs surface.

For this description, antimicrobial molecules (AM), Persulfate anions, are selected as the functional group; however, many different entities that need to be transported into a patient may be used. The Persulfate anions are from a stable Potassium Monopersulfate Triple salt. This is produced with increased active oxygen content from about 6.0% to about 7.5% using standard methods. The Potassium Monopersulfate Triple salt should be manufactured with substantially no K₂S₂O₈ for best results.

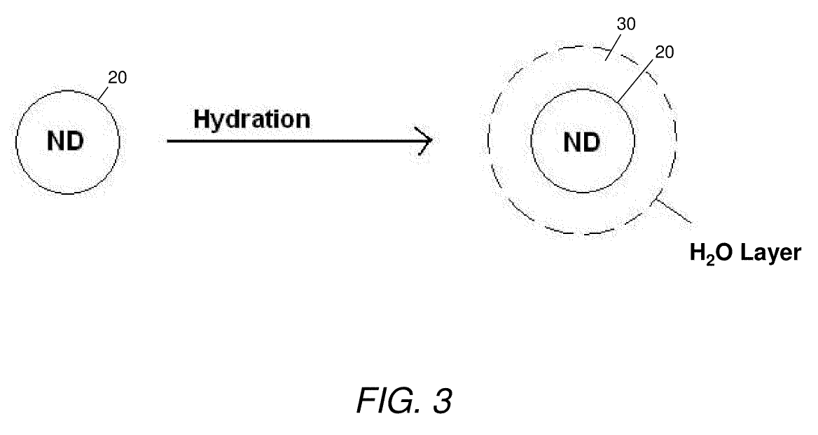

These steps of the present invention are schematically represented in FIGS. 3-6.

### 1. Hydration Step

In FIG. 3, the exposed surface of the NDs 20 is hydrated to create a water layer 30 on its surface.

Suspension of ND particles in the aqueous medium by sonication process generates electrostatic charges on the diamond surfaces. These charges attract water molecules to the diamond surface by hydration as shown as water layer 30 in FIG. 3. The electrostatic charges also provide the separation of the nanodiamond particles to achieve suspension. The NDs particles 20 are, in effect, solvated. These water molecules will also provide a pathway for ions to arrive at the diamond surface.

### 2. Intermediate Step

The electrostatic charges which attract the water molecules also attract ions to the surface. It is reasonable to assume that the smallest ions will be attracted to the diamond surface of nanodiamond particles (20 of FIG. 3) and will settle in the available sites.

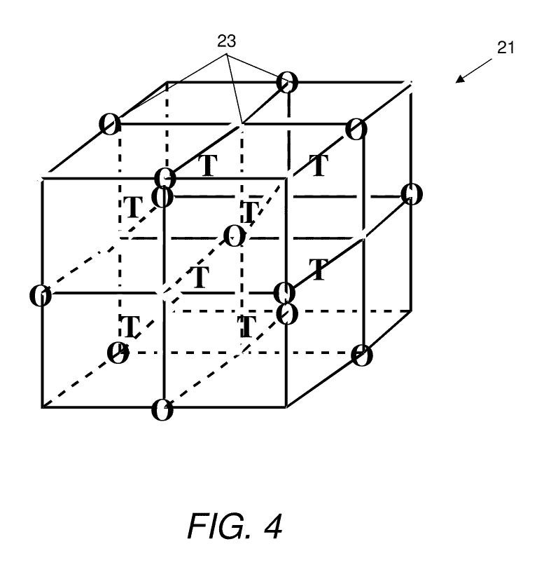

FIG. 4 shows carbon chain surface molecules 23 of a diamond cubic lattice structure 21 on the surface of a nanodiamond particle (20 of FIG. 3). The lattice 21 contains octahedral (O) and tetrahedral (T) interstices. When an ionic solution is provided to the nanodiamond particles 20, cation ions embed in the lattice 21 first, followed by anions. Cations are smaller than anions and will embed into the lattice 21 more easily. This causes crystal growth on the surface of the diamonds.

Incorporation of Cations into Nanodiamond Structure

The octahedral interstices indicated by “O” in FIG. 4, available in the lattice 21 have a radius of 101 pm, as calculated from the structure of diamond and the covalent radius of carbon atoms. All these octahedral interstices are empty and can accommodate species with radius smaller than 101 pm. The sonication of nanodiamonds for suspension in aqueous media generates electrostatic charges on the surfaces of the nanodiamond particles and these electrostatic charges are assumed to attract cations to the diamond particles. In order to optimize the efficacy and stability of the final AM functionalized NDs, the kind of cations selected to be accommodated into these octahedral interstices is critical. For the sake of illustration, the ionic radii for the cations of the first transition series are shown in Table 1.

TABLE 1
Ionic Radii for Cations of the First Transition Series Elements

| | Element | | | | | | | | | | | | | | | |
|---|---|---|---|---|---|---|---|---|---|---|---|---|---|---|---|---|
| | Sc | Ti | V | Cr | Mn | Mn | Fe | Fe | Co | Co | Ni | Cu | Cu | Ag | Na | K |
| Charge | +3 | +3 | +3 | +3 | +2 | +3 | +2 | +3 | +2 | +3 | +2 | +1 | +2 | +1 | +1 | +1 |
| Radius (pm) | 81 | 76 | 74 | 69 | 80 | 66 | 76 | 64 | 74 | 63 | 72 | 96 | 72 | 108 | 95 | 133 |
| Charge Density | | | | | | | | | | | | | | | 0.28 | 0.10 |

This suggests that any first transition metal ion can be incorporated into the octahedral interstices “O” of the diamond lattice 21. It should also be recognized that all of these metals have insoluble hydroxide compounds, some rather complicated as hydrated oxides. They will coordinate with a variety of donor atoms, such as oxygen, nitrogen, and sulfur, which are generally atoms included in AM, proteins, peptides, etc.

### Cation Selection for Stable Persulfate Deposition on NDS

As the cations are incorporated into the nanodiamond surfaces, the water layers adsorbed onto the diamond surfaces should be released. However, these transition metal ions will include water molecules on the cations with the stoichiometry M (H₂O)₆⁺ⁿ, where n is 2 or 3.

The information for Sodium and Potassium is incorporated into Table 1 in order to clarify several aspects of the crystal growth in the Potassium Persulfate deposition. Sodium ions were used since it is clear that Na⁺ will fit into the octahedral interstices of the diamond structure better than will the K⁺ ions. The major reason is that Sodium salts are hydrated because the Na⁺ is not large enough to pack efficiently into the crystal structure for larger anions, such as HSO₄⁻ and HSO₅⁻. In order to stabilize these structures, water molecules are incorporated to fill up space. The K⁺ ions, on the other hand, are well known to form anhydrous and very stable salts with larger anions, such as the bisulfate and Persulfate. Therefore, if we were to use NaOH to neutralize the acidity from the sonicated nanodiamonds, we might encourage the incorporation of water molecules into the crystal growth on the nanodiamonds, and this could lead to easy and rapid decomposition as the proton from the HSO₄⁻ could easily find a pathway to the peroxy group, thereby causing decomposition.

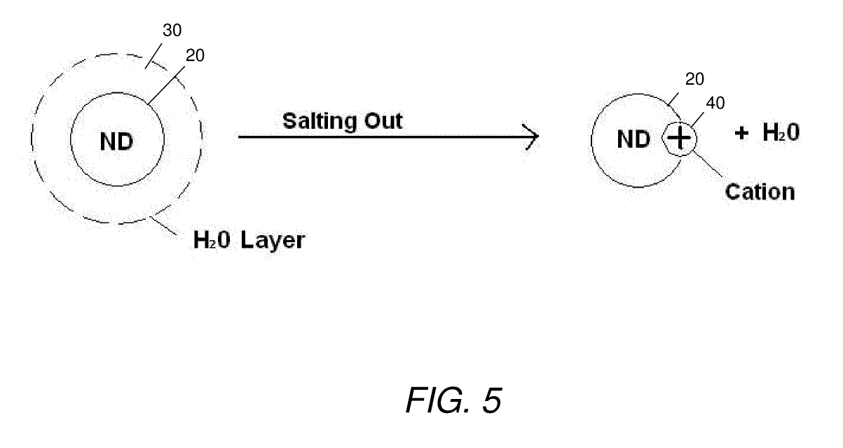

FIG. 5 illustrates the deposition of the cations onto the diamond surface. The deposition of cations results in crystalline growth on the surface of the NDs. This crystalline growth will displace the water molecules from the ND 20 surface, ‘salting out’ water molecules as the crystal growth continues on the surface of ND 20. In effect, ND 20 are utilized as nucleation sites for crystal growth.

Synthesis of ND with Cation Functionality Required for Ionic Crystal Growth

The normal infrared spectrum of NDs 20 is shown in Table 3. This will be compared later with the resultant functionalized NDs 54.

5.0028 g of nanodiamonds was placed in a 2 L beaker with 1 L of distilled water. The beaker was placed in an ice bath and the mixture was sonicated at a power output of 0.08 watts for about 0.5 hours. The temperature of the mixture was now 5 C. 0.01M KOH solution was also placed in the ice bath to cool them before use. Culled portions of the 0.01M KOH (Cation—K+) were added to the sonicated nanodiamond mixture until the pH was in the 6.95 to 7.05 range. The resultant mixture was sonicated at a power output of 0.08 watts for about 0.5 hours. The temperature of the mixture was now 5 C.

This present cation substitution on NDs 20 is shown in FIG. 5. The NDs now have cations 40 which are electrostatically attached to the surface of the NDs 20.

### 3. Functionalization Step

The resultant mixture was sonicated for an additional 15 to 20 minutes. 500 mL of the cold (about 2 C) DuPont oxone solution (source of Persulfate anion) was added to initiate the ionic crystal growth. The mixture was stirred with a thermometer. The temperature of the mixture was now about 1 C. Precipitation began after a very few minutes. As the solid settled, the temperature rose to 2 C. About 1 L of supernatant liquid was removed as the precipitate settled. The remaining mixture was freeze dried. This procedure has allowed us to prepare nanodiamond/Persulfate materials with an active oxygen content of 6-7% on a reproducible basis.

The wet chemistry process proceeds under mild conditions which selectively creates the desired cations surface functionalities.

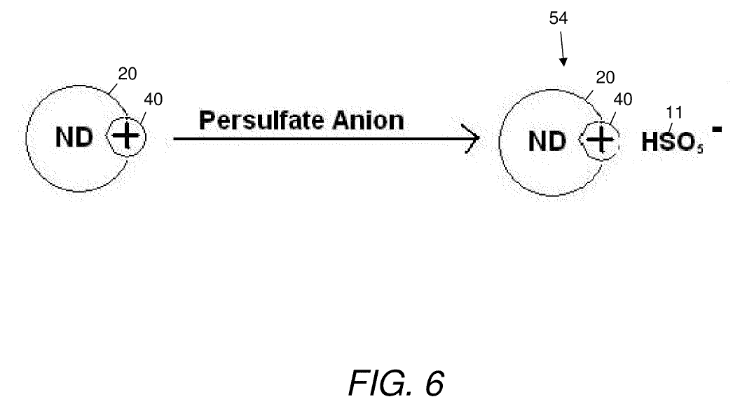

FIG. 6 is an illustration of functionalization of the surface of NDs 20 coating them with Persulfate anions, a functional group having antimicrobial properties. An ionic bond is created between the interstitial cations, indicated by “+” embedded within the ND 20 surface structure. This process results in functionalized ND particles 54 having the Persulfate functional groups coating the surface according to the present invention.

Active Oxygen Evaluation of AM

Below is a process for verification that the proper substance has been created.

In all examples active oxygen concentrations are expressed in weight % and are determined by standard Ioddometric titration as described in the “OXONE Monopersulfate Compound Technical information” bulletin, No H42434-5, published by E. I. du pont Nemours and company. Briefly, this measurement steps are defined below:

Take a representative sample by riffling, quartering, blending, or other means.

From the sample, carefully weigh at least two specimens of 0.4+/−0.001 g. each.

Add to a 250-mL beaker equipped with magnetic stirrer: 75 mL deionized water, 15 mL 10% (v/v) sulfuric acid, and 10 mL 25% (w/w) potassium iodide solution. (Deionized water and all reagents should be <20° C.). Add a weighed specimen and stir until dissolved.

Immediately titrate the specimen with 0.1 N sodium thiosulfate solution to a pale yellow color. Add 2 mL starch indicator solution, and the solution will turn deep blue. Immediately continue the titration to a colorless endpoint that persists for at least 30 sec.

### Calculations

% active oxygen=(mLthio·Nthio·0.008·100)/specimen weight (g)

% active component (KHSO₅)=% active oxygen/0.1053

Report the average of specimens analyzed.

### Active Oxygen Analysis of the Peroxysulfate Processed by Ionic Crystal Growth:

Samples 1-6 were processed based on steps identified for ionic crystal growth synthesis. When produced using the pH neutral, cold procedure active oxygen percentages were well above six. These results are shown in Table 2.

TABLE 2
Active Oxygen measurements for Peroxysulfate on nanodiamonds

| Sample | % Active Oxygen | PH of Analysis Mixture |
|---|---|---|
| 1 | 6.63 | 2.4 |
| 2 | 6.66 | 2.4 |
| 3 | 6.50 | 2.3 |
| 4 | 6.98 | 2.4 |
| 5 | 7.51 | 2.4 |

Each analysis is the average of three titrations on the sample. The PH values for the three titrations vary by approximately 0.1 PH unit.

As stated above, the active oxygen content should be preferably about 6.0% to about 7.5%.

There are several observations to be made based on these results. First, the amount of active oxygen has been considerably increased compared to % active Oxygen of starting materials OXONE solution with trade name, ACTXONE or Triple salt (% AO—3.5). Indeed the percentages of Active Oxygen measured in Peroxysulfate on nanodiamonds exceed the activities of reported values on commercial materials. This suggests that multiple layers of ionic material are being deposited onto the nanodiamond surfaces. Second, the analysis mixtures are highly acidic. This means that KHSO₅ is not the only material which precipitates on the nanodiamond surface; KHSO₄ must also be precipitating onto the diamonds. The acidity is not caused by the proton in KHSO₅ because the dissociation constant for that proton is only 5×10⁻¹⁰. Such a small dissociation constant cannot produce solutions with a PH between 2 and 3. A very simple calculation for a 0.1M solution of KHSO₅ suggests the PH of that solution is above five. Therefore, some KHSO₄ must be precipitating out with the KHSO₅. The dissociation constant for HSO₄⁻ is 1.0×10⁻² and a 0.1M solution of KHSO₄ will produce a PH in the vicinity of 1.5, much closer to the values shown in Table 2. Third, Active Oxygen measurements of Peroxysulfate on nanodiamond over five months period, suggest the stability of the product is increased extensively, as well. The synthesized Peroxysulfate on nanodiamond were placed in a resealable plastic bag and then stored in plastic bucket with desiccant materials placed on the bottom of the bucket with closed air tight lid. The active oxygen content of synthesized and packaged Peroxysulfate on nanodiamond were measured over the five months indicates the loss of less than %1 AO/month. It is necessary to indicate that synthesized Peroxysulfate on nanodiamond material has not been mixed with any anti caking agent.

Fourth, the nanodiamond-peroxysulfate infrared spectrum adduct was examined using attenuated total reflectance spectroscopy. A Nicolet 4700 FT-IR equipped with a SMART MIRacle ATR accessory was used to take the spectra. A sample spectrum is shown in Table 3 and compared directly with the same type of spectrum taken for the unreacted nanodiamonds.

TABLE 3
Infrared Spectrum of Nanodiamonds-peroxysulfate adduct.

| | |
|---|---|
| Unreacted | Nanodiamond-Peroxysulfate |
| Nanodiamonds | Adduct |

| Observed | | Observed Peak | |
|---|---|---|---|
| Peak (cm⁻¹) | Absorbance | (cm⁻¹) | Absorbance |
| 3406 | 0.0135 | 3257 | 0.12 |
| 1721 | 0.00574 | 2817 | 0.04 |
| 1656 | 0.00963 (sh) | 1631 | 0.02 |
| 1639 | 0.0117 | 1433 | 0.05 |
| 1097 | 0.0285 | 1295 | 0.16 (sh) |
| | | 1230 | 0.55 |
| | | 1071 | 0.34 |
| | | 1058 | 0.62 |
| | | 879 | 0.16 |

The unreacted nanodiamond spectrum is included here for comparison purposes.

In order to verify the nanodiamond-peroxysulfate absorbances, it is necessary to assign these peaks to specific vibrations in the sample. Table 4 contains assignments which seem most reasonable.

TABLE 4
Infrared Spectral Assignments for the
Nanodiamond-Peroxysulfate Peaks.

| Observed | |
|---|---|
| Peaks | Assignment |
| 3257 | Still in the OH range, but a different OH from that for the |
| | unreacted nanodiamonds. The OH from the peroxide |
| | group in the peroxysulfate. |
| 2817 | This is in the C—H stretching frequency range and is |
| | probably due to the C—H groups on the |
| | nanodiamond surface. |
| 1631 | C—O stretching frequency from the COH groups |
| | on the surface of the nanodiamonds. |
| 1295 | asymmetric bending frequency from the peroxy group. |
| 1230 | asymmetric bending frequency from the peroxy group. |
| 1071 | Possible ν₃ vibration from the SO₄⁼ group |
| 1058 | S—O stretching frequency from the HSO₅⁻ group. |
| 879 | symmetric O—O bending frequency from the peroxy |
| | group. |

References used to make these assignments are: (1) K. Nakanishi, Infrared Absorption Spectroscopy, Holden-Day, Inc., San Francisco (1962); (2) F. A. Cotton,

The Infra-red Spectra of Transition Metal Complexes, in Modern Coordination Chemistry, Edited by J. Lewis and R. G. Wilkins, Interscience Publishers Inc., New York (1960); (3) D. H. Williams and I. Fleming, Spectroscopic Methods in Organic Chemistry, McGraw-Hill Ltd, New York (1987).
Synthesis of ND with Mono-Layer AM-Silver

In an alternative embodiment, the functional group will be elemental silver instead of Peroxysulfate as described above.

1.0023 g of nanodiamonds were placed in 200 mL of distilled water and sonicated for one-half an hour in an ice bath. 17.0058 g of AgNO₃ were dissolved in 100 mL of distilled water. 83 mL of the silver nitrate solution was added to the sonicated nanodiamond suspension. Precipitation began at once. Approximately 200 mL of supernatant liquid was removed and the remaining mixture was freeze dried.

### Kirby-Bauer Antibiotic Testing

Kirby-Bauer antibiotic testing (KB testing or disk diffusion antibiotic sensitivity testing) is a test which uses anti-biotic impregnated wafers to test whether particular bacteria are susceptible to specific antibiotics. Persulfate coated NDs 54 of FIG. 6 were used to impregnate the wafers. A known quantity of bacteria are grown on agar plates in the presence of thin wafers containing relevant antibiotics. If the bacteria are susceptible to a particular antibiotic, an area of clearing surrounds the wafer where bacteria are not capable of growing (called a zone of inhibition).

The rate of antibiotic diffusion is used to estimate the bacteria's sensitivity to that particular antibiotic. In general, larger zones correlate with smaller minimum inhibitory concentration (MIC) of antibiotic for that bacteria. This information can be used to choose appropriate antibiotics to combat a particular infection.

### Materials and Reagents Used.

Nanodiamond powder with phase purity higher than 97% and particle sizes ranging from 4-10 nm produced by detonation synthesis.

Silver Nitrate and KOH substances were purchased from Aldrich chemical.

Oxone solution with trade name, ACTXONE was purchased from Dupont Company.

Freeze drying of paste materials was done by labconco unit as well as commercially at LYOPHILIZATION TECHNOLOGY, INC.

A Nicolet 4700 FT-IR equipped with a SMART MIRacle ATR accessory was used to take the spectra.

### Antimicrobials

The functional group selected above was an antimicrobial, Persulfate. However, numerous drugs, agents and functional groups may be attached to the ND surface and receive the same increase in efficacy. A few examples are provided below, however the present invention is useful for a wide variety of drugs and substances that can be attached by this method to carbon chain surface molecules of nanodiamonds.

Another preferred antimicrobial intended to be used with the present invention is: peroxymonosulfate triple salt and antimicrobial/antifungal chemistries as described in U.S. Pat. Nos. 7,090,820 and 4,131,672 issued Aug. 15, 2006 and Jan. 26, 1978 respectively.

Some other anti-microbial agents that can be used with the present invention include Cipro (Phizer), fluoroquinilone, Amoxycillin, 2-bromo-2-nitropropane-1,3-diol (for example, Canguard® 409 made by Angus Chemical Co., Buffalo Grove, Ill. 60089) and 3,5-dimethyltetrahydro-1,3,5-2H-thiazine-2-thione (for example, Nuosept® S made by Creanova, Inc., Piscataway, N.J. 08855 or Troysan®. 142 made by Troy Chemical Corp., West Hanover, N.J. 07936).

Other solid anti-microbial agents include N-(trichloromethyl)-thiop-hthalimide (for example, Fungitrol® 11 distributed by Creanova, Inc.), butyl-p-hydroxy-benzoate (for example, Butyl Parabens®. made by International Sourcing Inc., Upper Saddle River, N.J. 07458), diiodomethyl-p-tolysulfone (for example, Amical®. WP made by Angus Chemical Co.), and tetrachloroisophthalonitrile (for example, Nuocide® 960 made by Creanova, Inc.).

Drugs such as azithromycin, penicillin, clarithromycin, etc can be bound to the nanodiamond to increase efficacy as well.

Metals such as silver, copper and zinc and their metal ions also have anti-microbial properties. Silver ions have widespread effect as an anti-microbial agent. For example, silver ions may be effective against bacteria such as Escherichia coli and Salmonella typhimurium, and mold such as Asperigillus.

Sources of silver for functional groups for anti-microbial use include metallic silver, silver salts and organic compounds that contain ionic silver. Silver salts may include for example, silver carbonate, silver sulfate, silver nitrate, silver acetate, silver benzoate, silver chloride, silver fluoride, silver iodate, silver iodide, silver lactate, silver nitrate, silver oxide and silver phosphates. Organic compounds containing silver may include for example, silver acetylacetonate, silver neodecanoate and silver ethylenediaminetetraacetate in all its various salts.

Silver containing zeolites (for example, AJ10D containing 2.5% silver as Ag(I), and AK10D containing 5.0% silver as Ag(I), both distributed by AgION™ Tech L.L.C., Wakefield, Mass. 01880) are of particular use. Zeolites are useful for functional groups because when carried in a polymer matrix they may provide silver ions at a rate and concentration that is effective at killing and inhibiting microorganisms without harming higher organisms.

Silver containing zirconium phosphate (for example, AlphaSan® C 5000 containing 3.8% silver provided by Milliken Chemical, Spartanburg, S.C. 29304) is also particularly useful. In general zirconium phosphates act as ion exchangers. However, AlphaSan® C 5000 is a synthetic inorganic polymer that has equally spaced cavities containing silver, wherein the silver provides the anti-microbial effects. Silver zirconium phosphates are typically incorporated into powder coatings between 0.1 and 10 percent by weight and particularly 0.5 to 5 percent by weight of the total powder coating formulation.

The antimicrobial drug entity 11 may also be a stable potassium monopersulfate triple salt. Preferably this is produced with increased active oxygen content from about 6.0% to about 7.5%. It is more effective if there is substantially no K₂S₂O₈.

Using AMs listed above in combination with NDs, the present invention greatly increases efficacy in fighting microbes. Below are microbes which may be reduced or stopped with greater efficacy.

Bacteria

The present invention, using nanodiamonds to enhance the effectiveness of the above-referenced AMs against bacteria such as:

Bacillus cereus, Campylobacter jejuni, Chlamydia psittaci, Clostridium perfringens, Listeria monocytogenes, Shigella sonnei, Streptococcus pyogenes, Helicobacter pylori, Campylobacter pyloridia, Klebsiella pneumoniae, Escherichia coli, Salmonella typhimurium, Salmonella choleraesuis, Pseudomonas aeruginosa, Staphylococcus aureus, MRSA (Methicillin-Resistant Staphlococcus aureus), Staphylococcus epidermidis and VRE (Vancomycin-Resistant Enterococci faecalis).

Viruses

It is also effective at increasing efficacy against viruses such as:

Hepatitis A (HAV), Hepatitis B (HBV), Hepatitis C(HCV), HIV-1 (AIDS Virus), Influenza A, Norovirus (Norwalk-like viruses) and RSV (Respiratory syncytial virus).

Fungi

It is also effective at increasing efficacy against fungi such as:

Candida albicans and Trichophytou mentagrophytes.

### Method of Delivery

There are various known methods of introducing the ND-AM complexes into the body of the patient. For example, the most obvious would be in a pill or liquid form which the subject ingests. This is only allowable for drugs which are not effected by the acids of the digestive tract.

The ND-AM complexes may injected, administered by air gun, nose spray, be inhaled, or used as a suppository.

The ND-AM complexes may be used as a disinfectant as an air spray, applied to the hands, or incorporated into materials around the patient, such as sheets and bedding.

They may also be incorporated into medical disposables, such as surgical drapes, bandages and disposable coverings.

The ND-AM complex may be used to coat the woven or non-woven fabrics. These may be for disposable or non-disposable fabrics. The ND-AM complexes may also be incorporated into the actual fibers used to create the woven or non-woven fabrics; and, includes but is not limited to applications in producing sutures, wound dressings, and medical cable coatings.

### Test Results

NDs coated with AMs showed exceptional results in fighting microbes. The coated NDs showed significantly enhanced efficacy when compared to the AMs used alone.

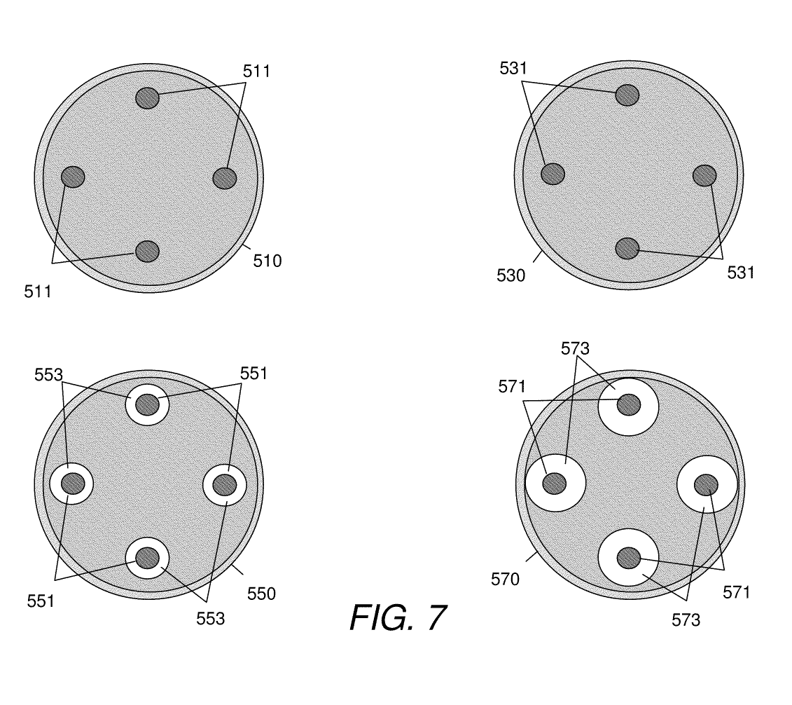

In FIG. 7 an illustration of the test results are shown. Three petri disks are shown filled with agar gel. Staphlococcus aureus was previously grown evenly across the surface of the gels.

Sixteen approximately ¼″ diameter discs were fabricated from of DuPont Sontara 8801 non-woven fabric.

Four disks 511 were placed on the surface of the agar in dish 510.

Four disks 531 were coated with nanodiamond powder (A of FIG. 3) and were placed on the surface of the agar in dish 530.

Four disks 551 were coated with potassium monopersulfate, the antimicrobial described above and were placed on the surface of the agar in dish 550.

Four disks 571 were coated with potassium monopersulfate used as the antimicrobial attached to, and covering the entire surface of the NDs being entity D of FIG. 5 described above and were placed on the surface of the agar in dish 570.

The plates were all please in the same environment overnight, then examined.

Plate 510, 530 had continuous growth of the bacteria and were not changed.

Plate 550 developed a clear area 553 around each disk 551 which was due to the destruction of the bacteria previous growing in this region. This area extended approximately 1/16″ away from each of the disks 551.

Plate 570 developed a clear area 573 around each disk 571 which was due to the destruction of the bacteria previous growing in this region. This area extended approximately ⅛″ away from each of the disks 551.

This indicates that the disks 511 and the disks with ND 531 did not have any effect on the microbes.

The antimicrobial on disks 553 killed bacteria a small distance away and the antimicrobial attached to ND on disks 575 killed bacteria a significantly larger distance away.

The surface are of a spherical surface increases surface area based upon the square of the radius. Therefore if a given number of molecules has to cover a larger surface area, the concentration drops proportionally. Therefore, the ND-AM complex kills at a significantly lower concentration. This shows increased antimicrobial efficacy.

Medical Foam

The nanodiamond enhanced particles shown as ND-AM entity 54 on FIG. 6, may be mixed with a thermoplastic, then pumped with air to create either closed cell, or open cell medical foam. As a liquid, the foam resembles suds. Many different known plastics may be used. For our example below, the foam was created from polyurethane. It was then hardened into a spongy-like foam material with antimicrobial properties. This foam will be useful as a medical wound dressing.

Animal Testing

Medical foam was created as indicated above then sent to a medical laboratory for testing. An animal study was performed on 20 mice by an independent lab testing authority.

This activated polyurethane foam was created with approximately almost 4% active material (ND-AM entity 54 on FIG. 6). The thickness of the used foam is 1.5 mm. This is referred to in the figures as the “Test Article”.

Wound areas having a diameter of about 4 mm. on each of the mice were infected by cultured Methycillin Resistant Staphlococcus Aureus (MRSA) infected with a 0.1 ml volume dose of inoculum which occurred only on Day 0.

There was also polyurethane foam without the ND-AM of the same size and thickness placed on the mice as a control for comparisons. This is referred to in the figures as the “Control”.

The foam was changed daily on each of the wounds, and the results tabulated.

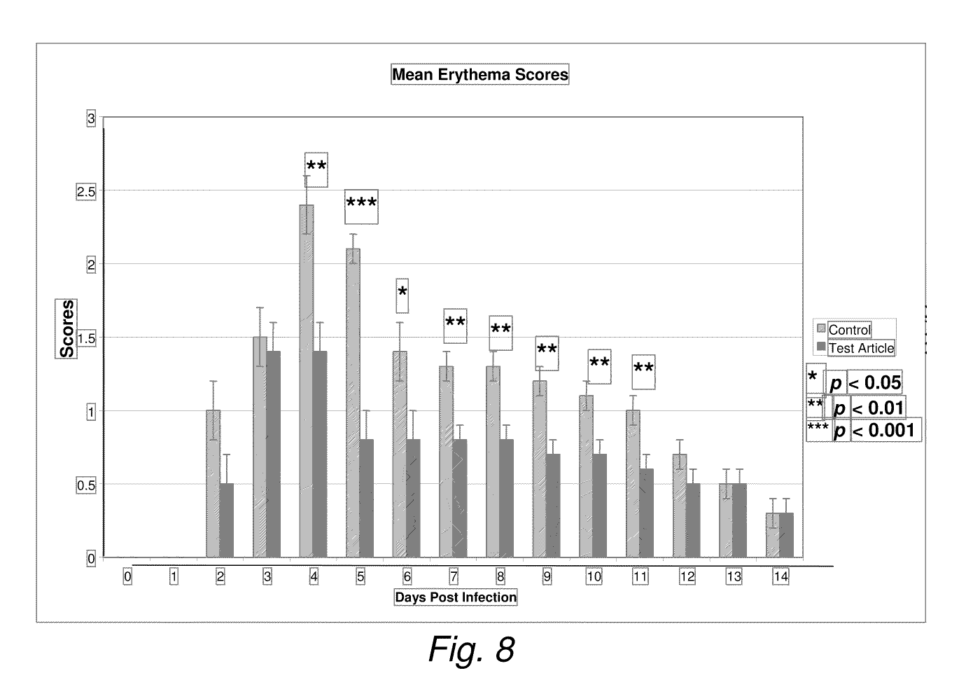

The data is listed below. Table A shows Mean Erythema Scores of both the tested group and the control group for consecutive 14 days. This is shown graphically in FIG. 8.

TABLE A
Mean Erythema Scores

| | | Erythema Scores | | | | | | | |
|---|---|---|---|---|---|---|---|---|---|
| | | (mean ± sem) | | | | | | | |
| Group | Treatment | Day 0 | Day 1 | Day 2 | Day 3 | Day 4 | Day 5 | Day 6 | Day 7 |
| 1 | Vehicle | 0 | 0 | 1.0 ± 0.2 | 1.5 ± 0.2 | 2.4 ± 0.2 | 2.1 ± 0.1 | 1.4 ± 0.2 | 1.3 ± 0.1 |
| 2 | Activated | 0 | 0 | 0.5 ± 0.2 | 1.4 ± 0.2 | 1.4 ± 0.2** | 0.8 ± 0.2*** | 0.8 ± 0.2* | 0.8 ± 0.2** |
| | PU foam | | | | | | | | |
| | | Day 8 | Day 9 | Day 10 | Day 11 | Day 12 | Day 13 | Day 14 | |
| 1 | Vehicle | 1.3 ± 0.1 | 1.2 ± 0.1 | 1.1 ± 0.1 | 1.0 ± 0.1 | 0.7 ± 0.1 | 0.5 ± 0.1 | 0.3 ± 0.1 | |
| 2 | Activated | 0.8 ± 0.1** | 0.7 ± 0.1** | 0.7 ± 0.1** | 0.6 ± 0.1** | 0.5 ± 0.1 | 0.5 ± 0.1 | 0.3 ± 0.1* | |
| | PU foam | | | | | | | | |

*Statistically significant difference compared to the Group 1 vehicle control (p < 0.05)
**Statistically significant difference compared to the Group 1 vehicle control (p < 0.05)
***Statistically significant difference compared to the Group 1 vehicle control (p < 0.05)

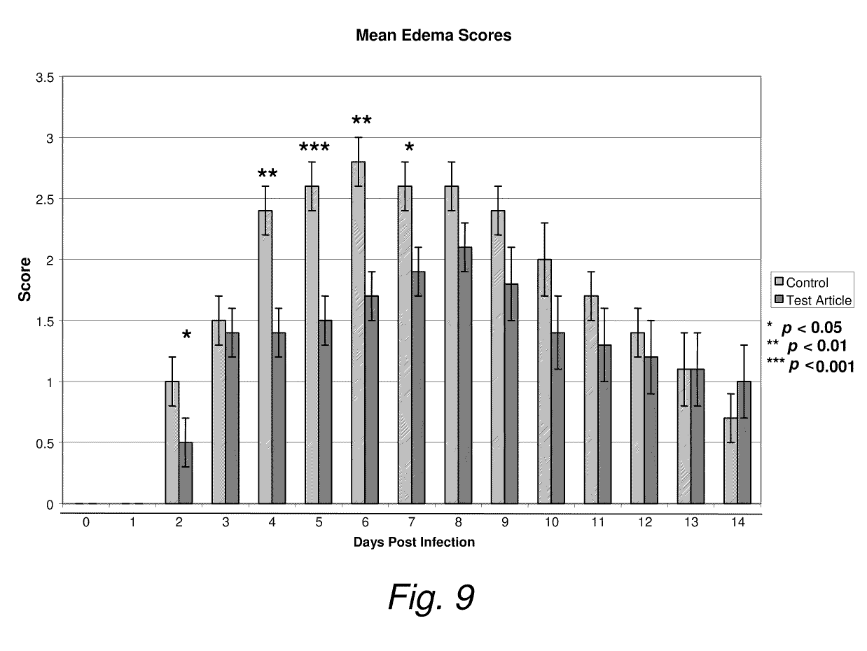

Table B shows the Mean Edema Scores for the same groups for 14 consecutive days. This is shown graphically in FIG. 9.

TABLE B
Mean Edema Scores

| | | Edema Scores | | | | | | | |
|---|---|---|---|---|---|---|---|---|---|
| | | (mean ± sem) | | | | | | | |
| Group | Treatment | Day 0 | Day 1 | Day 2 | Day 3 | Day 4 | Day 5 | Day 6 | Day 7 |
| 1 | Vehicle | 0 | 0 | 1.0 ± 0.2 | 1.5 ± 0.2 | 2.4 ± 0.2 | 2.6 ± 0.2 | 2.8 ± 0.2 | 2.6 ± 0.1 |
| 2 | Activated | 0 | 0 | 0.5 ± 0.2* | 1.4 ± 0.2 | 1.4 ± 0.2** | 1.5 ± 0.2*** | 1.7 ± 0.2** | 1.9 ± 0.2* |
| | PU foam | | | | | | | | |
| | | Day 8 | Day 9 | Day 10 | Day 11 | Day 12 | Day 13 | Day 14 | |
| 1 | Vehicle | 2.6 ± 0.2 | 2.4 ± 0.2 | 2.0 ± 0.3 | 1.7 ± 0.2 | 1.4 ± 0.2 | 1.1 ± 0.3 | 0.7 ± 0.1 | |
| 2 | Activated | 2.1 ± 0.2 | 1.8 ± 0.3 | 1.4 ± 0.3 | 1.3 ± 0.3 | 1.2 ± 0.3 | 1.1 ± 0.3 | 1.0 ± 0.3 | |
| | PU foam | | | | | | | | |

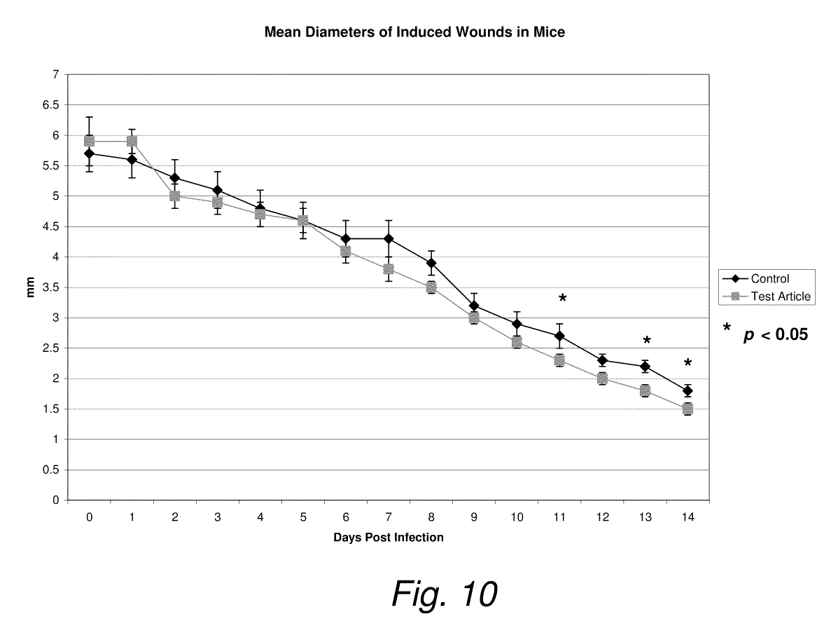

Table C shows the Mean Wound Diameters for the same groups for 14 consecutive days. This is shown graphically in FIG. 10.

TABLE C
Mean Wound Diameters

| | | Wound Diameters | | | | | | | |
|---|---|---|---|---|---|---|---|---|---|
| | | (mean ± sem) | | | | | | | |
| Group | Treatment | Day 0 | Day 1 | Day 2 | Day 3 | Day 4 | Day 5 | Day 6 | Day 7 |
| 1 | Vehicle | 5.7 ± 0.3 | 5.6 ± 0.3 | 5.3 ± 0.3 | 5.1 ± 0.3 | 4.8 ± 0.3 | 4.6 ± 0.3 | 4.3 ± 0.3 | 4.3 ± 0.3 |
| 2 | Activated | 5.9 ± 0.4 | 5.9 ± 0.2 | 5.0 ± 0.2 | 4.9 ± 0.2 | 4.7 ± 0.2 | 4.6 ± 0.2 | 4.1 ± 0.2 | 3.8 ± 0.2 |
| | PU foam | | | | | | | | |
| | | Day 8 | Day 9 | Day 10 | Day 11 | Day 12 | Day 13 | Day 14 | |
| 1 | Vehicle | 3.9 ± 0.3 | 3.2 ± 0.2 | 2.9 ± 0.2 | 2.7 ± 0.2 | 2.3 ± 0.2 | 2.2 ± 0.1 | 1.8 ± 0.1 | |
| 2 | Activated | 3.5 ± 0.1 | 3.0 ± 0.1 | 2.6 ± 0.1 | 2.3 ± 0.1* | 2.0 ± 0.1 | 1.8 ± 0.1* | 1.5 ± 0.1* | |
| | PU foam | | | | | | | | |

Table D shows the Average Time to Exfoliation for both groups.

TABLE D
Average Time to Exfoliation

| | | Exfoliation Time |
|---|---|---|
| | | (days) |
| Group | Treatment | Mean ± SEM |
| 1 | Vehicle | 9.9 ± 0.8 |
| 2 | Activated PU Foam | 10.8 ± 0.6 |

Table E shows the Body Weights of the test groups over the test period.

TABLE E
Body Weights

| | | Body Weight (g) | | |
|---|---|---|---|---|
| | | (mean ± sem) | | |
| Group | Treatment | Day 0 | Day 7 | Day 14 |
| 1 | Vehicle | 24.6 ± 0.4 | 25.2 ± 0.6 | 25.8 ± 0.6 |
| 2 | Activated PU Foam | 23.2 ± 0.4 | 23.9 ± 0.6 | 24.4 ± 0.7 |

Other Test Results are shown below.

Wound infection is a major complication in diabetic patients. According to the American Diabetes Association, 25% of people with diabetes will suffer from a wound problem during their lifetime, and it has been estimated that lower limb amputations in diabetic patients account for >60% of all amputations performed.

Staphylococcus aureus (S. aureus) is the most common single isolate (76%) in diabetic wounds and foot ulcers and leads to alterations in wound healing. Wound infection can also result in bacteremia or sepsis and is associated with high morbidity and mortality. In the United States S. aureus is the most common cause of skin and soft-tissue infections, as well as of invasive infections acquired within hospitals. Treatment of severe S. aureus infections is challenging, and the associated mortality rate remains high. S. aureus is a gram-positive bacterium that colonizes the skin and is present in the anterior nares in about 25-30% of healthy people. Over the last 40 years Methicillin-resistant S. aureus (MRSA) infections have become endemic in hospitals in the U.S. and worldwide. In 2002, the first clinical isolate of Vancomycin-resistant S. aureus (VRSA) was identified in a patient with diabetic foot ulcer. The progressive reduction of therapeutic efficacies of the available antibiotics underlines the need for the development of new therapeutic strategies for the treatment of infected wounds.

Therefore, the medical foam incorporating the enhanced antimicrobial would be very useful in countering the problems which diabetics encounter, especially since it was tested as effective against MRSA.

Even though this description was performed for a specific antimicrobial, it is believed that this applies to increasing the efficacy of other drugs and preparations. If these entities are used instead of the AM entities and attached to the surface of NDs, their efficacy will also increase.

A mentioned in the parent applications, the ND-drug complexes 54 can be added to resins used to manufacture fibers such as polypropylenes or other thermoplastics to make synthetic fiber materials. The fiber materials may be mixed with other natural fibers, or used by themselves. They can be added as fillers, pressed into non-woven materials, or woven into cloths.

These will all exhibit anti-microbial properties, such as resisting bacterial growth and odor as well as stopping fungal growth. Some of the non-woven disposable products are, for example diapers, incontinent products, sanitary napkins, surgical mask and other such a hygiene and personal care articles with improved antimicrobial properties.

The nanofibers or nanofiber nonwoven webs can be manufactured by conventional electrospinning processes. The fibers are in the range from about 0.04 to 2 micron in diameter with improved antimicrobial resistance.

Since other modifications and changes varied to fit particular operating requirements and environments will be apparent to those skilled in the art, the invention is not considered limited to the example chosen for the purposes of disclosure, and covers all changes and modifications which do not constitute departures from the true spirit and scope of this invention.

What is claimed is:

1. A method of enhancing efficacy of a drug entity 11 having an active site 13, comprising the steps of:
a) acquiring a plurality of nanodiamond (ND) particles 20 having a surface with a plurality of carbon chain surface molecules 23 in a crystalline lattice structure 21, the ND particles 20 having a diameter of less than 10 nanometers;
b) attaching water molecules 30 to the surface of the ND particles 20 to prepare said surface for further reactions;
c) embedding cations 40 within the lattice structure 21 of the surface molecules, creating crystalline growth;
d) providing said drug entity 11 in an anionic form to the surface of the ND particles 20 creating ionic bonds with the cations 40 embedded within the lattice structure 21, thereby coating the NDs particles 20 increasing efficacy and solubility of the drug functional groups 11.

2. The method of claim 1, wherein, the step of providing said drug entity 11 comprises:
selecting a drug entity 11 having an active site 13 that points away from the ND particle 20 when attached to the ND particle 20, thereby exposing the active sites for enhanced activity and enhanced drug efficacy.

3. The method of claim 1, wherein the drug entity 11 comprises:
antimicrobial agents.

4. The method of a claim 1 wherein the drug entity 11 comprises is the anion form of one of the group consisting of: fluoroquinilone, Amoxycillin, 2-bromo-2-nitropropane-1,3-diol, 3,5-dimethyltetrahydro-1,3,5-2H-thiazine-2-thione, N-(trichloromethyl)-thiop-hthalimide, butyl-p-hydroxy-benzoate, diiodomethyl-p-tolysulfone, and tetrachloroisophthalonitrile, azithromycin, penicillin and clarithromycin.

5. The method of claim 1, further comprising the step of:
administering the functionalized ND particles 54 to a patient by one of the group consisting of: injection, compressed air gun, nose spray, suppository.

6. The method of claim 1, further comprising the step of:
incorporating the functionalized ND particles 54 into threads of one of the group consisting of: woven fabrics used in medical industry, clothing having antimicrobial properties, and clothing resistant to microbial growth and unpleasant odors.

7. The method of claim 1, further comprising the step of:
incorporating the functionalized ND particles 54 into fibers of nonwoven fabrics of the medical industry used in one of the group consisting of: surgical drapes, disposable surgical garments, disposable wound care dressings.

8. The method of claim 1, further comprising the step of:
incorporating the functionalized ND particles 54 into a liquid plastic;
foaming the liquid plastic and functionalized ND particles 54 allowing the liquid plastic and ND particles (D) to harden into a medical foam adapted for use in wound care.

9. An enhancing efficacy drug entity 11 having an active site 13, created from the process comprising the steps of:
a) acquiring a plurality of nanodiamond (ND) particles 20 having a surface with a plurality of carbon chain surface molecules 23 in a crystalline lattice structure 21, the ND particles 20 having a diameter of less than 10 nanometers;
b) attaching water molecules 30 to the surface of the ND particles 20 to prepare said surface for further reactions;
c) embedding cations 40 within the lattice structure 21 of the surface molecules, creating crystalline growth;
d) providing said drug entity 11 in an anionic form to the surface of the ND particles 20 creating ionic bonds with the cations 40 embedded within the lattice structure 21, thereby coating the NDs particles 20 increasing efficacy and solubility of the drug functional groups 11.

10. The enhancing efficacy drug entity 11 of claim 9, wherein, the step of providing said drug entity 11 comprises:
selecting a drug entity 11 having an active site 13 that points away from the ND particle 20 when attached to the ND particle 20, thereby exposing the active sites for enhanced activity and enhanced drug efficacy.

11. The enhancing efficacy drug entity 11 of claim 9, wherein the drug entity 11 comprises:
antimicrobial agents.

12. The enhancing efficacy drug entity 11 of claim 9, wherein the drug entity 11 comprises than anion of one of the group consisting of:
fluoroquinilone, Amoxycillin, 2-bromo-2-nitropropane-1,3-diol, 3,5-dimethyltetrahydro-1,3,5-2H-thiazine-2-thione, N-(trichloromethyl)-thiop-hthalimide, butyl-p-hydroxy-benzoate, diiodomethyl-p-tolysulfone, and tetrachloroisophthalonitrile, azithromycin, penicillin and clarithromycin.

13. The enhancing efficacy drug entity 11 of claim 9, further comprising the step of:
incorporating the functionalized ND particles 54 into nanofiber threads of one of the group consisting of: woven fabrics used in medical industry, clothing having antimicrobial properties, and clothing resistant to microbial growth and unpleasant odors.

14. The enhanced efficacy drug entity 11 of claim 9 further comprising the step of:
incorporating the functionalized ND particles 54 into nanofibers nonwoven webs manufactured by electrospinning process, the webs being in the range of about 0.04 to 2 micron in diameter and exhibiting improved antimicrobial resistance.

15. The enhancing efficacy drug entity 11 of claim 9, further comprising the step of:
incorporating the functionalized ND particles 54 into fibers of nonwoven fabrics used in one of the group consisting of: surgical drapes, disposable surgical garments, disposable wound care dressings, disposable diapers, incontinent products, sanitary napkins, wearable mask and other such a hygiene and personal care articles with improved antimicrobial properties.

16. The enhancing efficacy drug entity 11 of claim 9, further comprising the step of:
incorporating the functionalized ND particles 54 into a liquid plastic;
foaming the liquid plastic and functionalized ND particles 54 allowing the liquid plastic and ND particles (D) to harden into a medical foam adapted for use in wound care.

17. A method of enhancing the efficacy of a drug entity 11 comprising the steps of:
a) acquiring nanodiamond (ND) particles having carbon chain surface molecules created by a detonation process with the majority of the particles having a diameter of less than 10 nm;
b) processing the surface of the ND particles 20 by hydrating them to create a layer of water molecules on the ND particles 20;
c) embedding cations within the carbon chain surface molecules thereby ‘salting out’ water molecules on the surface of the ND particles 20;
d) providing the drug entity 11 in anionic form to the surface of the ND particles 20 to cause an ionic bond with the cations 40 embedded within the carbon chain surface molecules 23 to result in functionalized ND particles 54 exhibiting enhanced efficacy when compared to prior art drugs.

18. The method of claim 17, wherein, the step of providing the drug entity 11, comprises the steps of:
selecting a drug entity 11 having an ionic attachment point substantially opposite its active site, such that when it is attached to the cation, the active site of said drug molecules 11 points away from the ND particle 20 surface exposing it enhanced activity creating enhanced drug efficacy.

19. The method of claim 17, wherein, the step of providing the drug entity 11, comprises the steps of:
providing potassium monopersulfate triple salt with active oxygen content from about 6.0% to about 7.5% by weight with substantially no K₂S₂O₈.

20. The enhancing efficacy drug entity 11 of claim 9, further comprising the step of:
incorporating the functionalized ND particles 54 into fibers of thread to make woven fabrics used in one of the group consisting of: surgical drapes, disposable surgical garments, diapers, wearable mask and other such a hygiene and personal care articles with improved antimicrobial properties.
